(19) United States
(12) Plant Patent Application Publication
(10) Pub. No.: US 2003/0233688 P1
(43) Pub. Date: Dec. 18, 2003

(54) Rose plant named 'Prerarust'

(75) Inventor: Theodorus A. Segers, Hoofddorp (NL)

Correspondence Address: Catherine Anne Whealy, Proprietary Rights International, 1513 Melody Lane, Roanoke, TX, 76262, US

(73) Assignee: Prego Royalty B.V., Naaldwijk (NL)

(21) Appl. No.: 10/171,829

(22) Filed: Jun. 17, 2002

(51) Int. Cl. A01H 5/00

(52) U.S. Cl. PLT/136

(57) ABSTRACT

A distinctive cultivar of Hybrid Tea Rose plant named ‘Prerarust’, characterized by its shiny dark green leaves; long, strong and green flowering stems; large salmon orange-colored flowers; and excellent postproduction longevity.

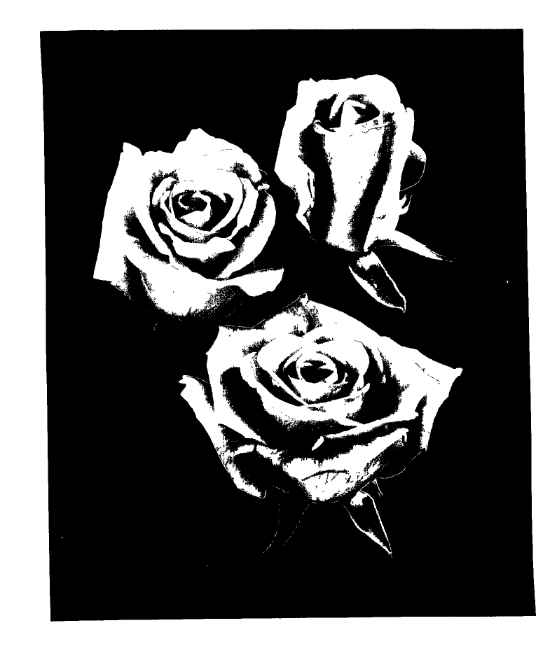

### BOTANICAL CLASSIFICATION/CULTIVAR DESIGNATION

Rosa hybrida cultivar Prerarust

### BACKGROUND OF THE INVENTION

The present invention relates to a new and distinct cultivar of Hybrid Tea Rose plant, botanically known as Rosa hybrida, and hereinafter referred to by the cultivar name Prerarust.

The new cultivar is a product of a planned breeding program conducted by the Inventor in Rijsenhout, The Netherlands. The objective of the breeding program was to develop new cut Rose cultivars with attractive flower colors, long and strong stems, dark green leaves, good production during the winter, and good postproduction longevity.

The new cultivar originated from a cross made by the Inventor in 1995 of two proprietary selections. The proprietary selection identified as code number 92-184, not patented, was the female, or seed, parent and the proprietary selection identified as code number 92-308, not patented, was the male, or pollen, parent. The cultivar Prerarust was discovered and selected by the Inventor as a flowering plant within the progeny of the stated cross in a controlled environment in Rijsenhout, The Netherlands.

Asexual reproduction of the new cultivar by cuttings taken at Rijsenhout, The Netherlands, has shown that the unique features of the new cultivar are stable and reproduced true to type in successive generations of asexual reproduction.

### SUMMARY OF THE INVENTION

The following traits have been repeatedly observed and are determined to be the unique characteristics of ‘Prerarust’. These characteristics in combination distinguish the new Hybrid Tea Rose as a new and distinct cultivar:

1. Shiny dark green leaves.

2. Long, strong and green flowering stems.

3. Large salmon orange-colored flowers.

4. Excellent postproduction longevity.

Compared to plants of the female parent, the selection 92-184, plants of the new Hybrid Tea Rose have shorter flowering stems, are more freely flowering, have longer postproduction longevity, are more resistant to Powdery Mildew and differ in flower color as plants of the selection 92-184 have orange-colored flowers. Compared to plants of the male parent, the selection 92-308, plants of the new Hybrid Tea Rose are more resistant to Powdery Mildew and differ in flower color as plants of the selection 92-308 have yellow-colored flowers.

Plants of the Hybrid Tea Rose can be compared to plants of the cultivar Pretaner, disclosed in U.S. Plant Pat. No. 11,272. In side-by-side comparisons conducted by the Inventor in Rijsenhout, The Netherlands, plants of the new Hybrid Tea Rose differed from plants of the cultivar Pretaner in the following characteristics:

1. Plants of the new Hybrid Tea Rose were more freely flowering than plants of the cultivar Pretaner.

2. Plants of the new Hybrid Tea Rose and the cultivar Pretaner differed in flower color as plants of the cultivar Pretaner had yellow and white-colored flowers.

### BRIEF DESCRIPTION OF PHOTOGRAPH

The accompanying colored photograph illustrates the new Hybrid Tea Rose plant, showing the colors as true as it is reasonably possible to obtain in colored reproductions of this type. Colors in the photograph may differ slightly from the color values cited in the detailed botanical description which more accurately describe the actual colors of the new Hybrid Tea Rose. The photograph comprises a side perspective view of typical flowers and leaves of the new Hybrid Tea Rose grown in Rijsenhout, The Netherlands.

### DETAILED BOTANICAL DESCRIPTION

Plants of the new Hybrid Tea Rose have not been observed under all possible environmental conditions. The phenotype may vary significantly with variations in environment such as temperature and light without, however, any variance in genotype.

The aforementioned photographs, following observations and measurements describe plants grown in Rijsenhout, The Netherlands, in glass-covered greenhouses with average day temperatures of 18° C. and average night temperatures of 17° C. Cut flowering stems used for the photographs and the description were about seven weeks old. In the following description, color references are made to The Royal Horticultural Society Colour Chart except where general terms of ordinary dictionary significance are used.

Classification:

Botanical.—Rosa hybrida cultivar Prerarust.

Commercial.—Hybrid Tea Rose used as a cut flower.

Parentage:

Female, or seed, parent.—Proprietary selection of Rosa hybrida identified as code number 92-184, not patented.

Male, or pollen, parent.—Proprietary selection of Rosa hybrida identified as code number 92-308, not patented.

Propagation:

Type.—By vegetative cuttings.

Time to initiate roots.—About 12 days at 25° C.

Time to develop roots.—About 25 days at 25° C.

Root description.—Fine, fibrous and well-branched.

Plant description:

Form.—Upright; narrow; bushy.

Plant height.—About 100 cm.

Plant width.—About 40 cm.

Growth habit.—Moderately vigorous.

Flowering stems.—Quantity per plant: About 3 or 4 per plant. Length: About 85 cm. Diameter: About 1.25 cm. Internode length: About 5 cm. Texture: Glabrous; with thorns. Color: 146A. Thorns: Amount: Abundant. Height: About 7 mm. Diameter, at base: About 8 mm. Shape: Triangular. Color: 177B.

Foliage description.—Arrangement: Alternate; compound with five to seven leaflets. Quantity per flowering stem: About 13 compound leaves. Leaf length: About 16 cm. Leaf width: About 12 cm. Terminal leaflet length: About 8.5 cm. Terminal leaflet width: About 5.5 cm. Leaflet shape: Ovate. Leaflet apex: Acute. Leaflet base: Obtuse. Leaflet margin: Serrate. Leaflet texture: Smooth, shiny. Leaflet venation pattern: Pinnate. Petiole length: To base of lower leaflet: About 3 cm. To base of terminal leaflet: About 9 cm. Petiole diameter: About 2.5 mm. Stipules: Two at either side of petiole base; less than 1 cm in length and about 1 mm in diameter; linear in shape. Color: Young foliage, upper surface: 200B. Young foliage, lower surface: 183A. Fully expanded foliage, upper surface: 139A. Fully expanded foliage, lower surface: 147B. Venation, upper surface: 139A. Venation, lower surface: 152A. Petiole and stipules: 137B.

Flower description:

Flower type and habit.—Large salmon orange-colored flowers. Consistently symmetrical rosette flowers. Freely and recurrent flowering habit. Flowers arranged singly at terminal apices or in sprays of 2 to 4 flowers and flower buds per spray. Flowers persistent.

Flowering season/time to flower.—Year-round under greenhouse conditions. Depending on environmental conditions and season, time to flower is about seven weeks.

Flower diameter.—About 12 cm.

Flower depth (height).—About 5.5 cm.

Flower longevity on plant.—About 17 days.

Flower longevity as a cut flower.—About 12 days.

Fragrance.—Not detected.

Flower buds (at stage of showing color).—Shape: Ovoid. Length: About 5 cm. Diameter: About 3.6 cm. Color: 42C.

Petals.—Quantity: About 35 per flower arranged in about 6 whorls. Length: About 6 cm. Width: About 7 cm. Shape: Orbicular. Apex: Mucronate. Margin: Entire, undulate, reflexed. Texture: Smooth, satiny. Color: When opening, upper surface: 48A. When opening, lower surface: 50C. Fully opened, upper surface: 37A; color fading to 37C with pale yellow, close to 10D, towards the margins with subsequent development. Fully opened, lower surface: 37B.

Sepals.—Quantity per flower: Five. Calyx length: About 4.6 cm. Calyx diameter: About 4.3 cm. Shape: Elongated, linear. Apex: Aristate. Margin: Mostly entire with random linear extensions, usually less than 1.5 cm in length. Texture: Smooth. Color, upper and lower surfaces: 146B.

Peduncles.—Strength: Strong. Aspect: Erect. Length: About 14 cm. Color: 146A.

Reproductive organs.—Stamens: Quantity per flower: About 50. Anther length: About 4 mm. Anther shape: Ovate. Anther color: 15B. Pollen amount: Abundant. Pollen color: 10B. Pistils: Quantity per flower: About 60. Style length: About 8 mm. Style color: 157D. Stigma color: 11A. Ovary color: 155B.

Seed.—Length: About 7 mm. Diameter: About 4.9 mm. Shape: Oval. Color: 159A.

Disease/Pest resistance: Resistance to Powdery Mildew has been observed on plants grown under commercial greenhouse production conditions. Plants of the new Hybrid Tea Rose have not been observed to be resistant to pests common to roses.

It is claimed:

1. A new and distinct Hybrid Tea Rose plant named ‘Prerarust’, as illustrated and described.
